(19) United States
(12) Patent Application Publication
(10) Pub. No.: US 2011/0123623 A1
(43) Pub. Date: May 26, 2011

(54) RHAMNOLIPID MECHANISM

(76) Inventor: Keith DeSanto, St. Petersburg, FL (US)

(21) Appl. No.: 12/938,985

(22) Filed: Nov. 3, 2010

Related U.S. Application Data
(60) Provisional application No. 61/257,541, filed on Nov. 3, 2009.
(60) Provisional application No. 61/285,562, filed on Dec. 11, 2009.

(51) Int. Cl. A61K 31/7084; A61K 9/14; A61P 17/00; A61P 29/00

(52) U.S. Cl. 424/489; 514/25

(57) ABSTRACT

A topical composition for treating a patient having rhamnolipid as an active ingredient. The rhamnolipid is absorbed by the skin of a human or animal, absorbed by the blood stream, and distributed through the human or animal body.

### REFERENCE TO RELATED APPLICATIONS

This application claims the benefit of U.S. Provisional Application No. 61/257,541 filed Nov. 3, 2009 and U.S. Provisional Application No. 61/285,562 filed Dec. 11, 2009, the contents of which are incorporated herein by reference.

### FIELD OF THE INVENTION

The present invention relates to rhamnolipids, and more specifically to the ability of the rhamnolipids to be absorbed into the blood stream when topically applied into the skin. In addition, the present invention relates to the use of rhamnolipids for the treatment of radiation burns.

### BACKGROUND OF THE INVENTION

The skin is one of the major routes in which chemicals enter the body. The skin consists of three main layers: the epidermis, the outer layer that you can see and touch; the dermis, the inner layer where all the work takes place; and subcutaneous fat, which separates the skin from the rest of the tissue in your body.

The epidermis consists of an outer layer of dead cells called keratinocytes, which provide a tough protective coating, and several layers of rapidly dividing cells just beneath the keratinocytes. The dermis lies immediately beneath the epidermis and is composed of cells called fibroblast, fibrous proteins (collagen, reticulum and elastin) and a material called ground substance. The dermis is the layer responsible for the skin's structural integrity, elasticity and resilience. Unlike the epidermis, the dermis contains blood vessels, which provide it with nutrients and oxygen and remove metabolic waste products. The dermis also contains lymphatic vessels and nerves, as well as specialized structures called appendages: sweat glands, sebaceous (oil) glands and hair follicles. (FIG. 1).

Collagen gives the skin its flexibility and provides structural support. The fibroblasts that make collagen are the main type of cells in the dermis. The ground substance, a composition of water and a variety of chemical substances, forms a matrix in which all other dermal components are embedded and through which the transfer of substances between cells occurs.

Normally, the epidermis and the dermis exist in steady-state equilibrium, forming a protective barrier against the external environment. Once the protective barrier is broken, the normal (physiologic) process of healing is immediately set in motion to repair the damage. Healing is divided into four sequential phases: 1) hemostasis, 2) inflammatory, 3) proliferative, and 4) remodeling.

Within minutes of the jury, platelets are present at the place of the injury to form a fibrin clot controlling the bleeding (hemostasis). In the inflammatory phase, bacteria and debris are removed causing the migration and division of the cells involved in the proliferative phase.

The proliferative phase is characterized by angiogenesis, collagen deposition, granulation tissue formation, epithelialization, and wound contraction. In angiogenesis, new blood vessels are formed. In fibroplasia and granulation fibroblasts grow and form a new, provisional extracellular matrix by excreting collagen and fibronectin. Then, re-epithelialization of the epidermis occurs; in which epithelial cells proliferate and cover the wound superficie.

As can be seen from above, all of the activity takes place on the dermis. Therefore, restoration treatment only has a chance to succeed if the composition can reach as deep as the dermis.

Unfortunately, many topical compositions never reach the dermis because the molecules of the ingredients are too large to penetrate the epidermis forming a film on the skin.

In recent years, the number of radiation burns has increased considerably. Radiation burns are caused by UV-lights, X-rays, Gamma radiation, microwaves, or sunlight, which alter tissue cell structures. Sometimes, the injury extends into the dermis, leaving few viable epidermal cells. Re-epithelialization is very slow and wounds require months to heal.

As can be seen, there is a need for a topical composition that can be absorbed by the blood stream on the dermis layer of the skin and at the same time has unique characteristics that could successfully repair the deep layer. Furthermore, there is a need for a topical composition that can be absorbed by the blood stream and at the same time promotes the skin repair in view of radiation burn injuries.

### SUMMARY OF THE INVENTION

The present invention relates to a compostion for treating a patient comprising as active ingridient a rhamnolipid of formula I,

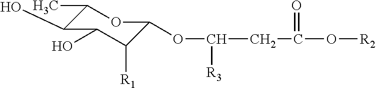

wherein R¹=H, unsubstituted α-L-rhamnopyranosyl, α.-L-rhamnopyranosyl substituted at the 2 position with a group of formula —O—C(═O)—CH═CH—R₅, or —O—C(═O)—CH═CH—R₅;
R²═H, lower alkyl, —CHR₄—CH₂—COOH or —CHR₄—CH₂—COOR₆;
R³=—(CH₂)ₓ—CH₃, wherein x=4-19;
R⁴=—(CH₂)_{y}—CH₃, wherein y=1-19;
R⁵=(CH₂)_{z}—CH₃, wherein z=1-12;
R⁶=lower alkyl;
wherein the composition is a topical composition that is absorbed by the skin of a human or animal, absorbed by the blood stream, and distributed through the human or animal body by the blood stream.

The rhamnolipid of formula I has a concentration of between 1-100 μg/ml and molecules having a size of about 1 micron.

The rhamnolipid of Formula I is α-L-rhamnopyranosyl-(1,2)-α-L-rhamnopyranosyl)-3-hydroxydecanoyl-3-hydroxydecanoic acid having the following formula:

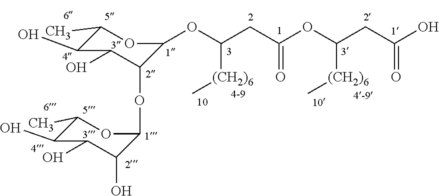

One or more rhamnolipids of Formula I are selected from the group consisting of compounds of Formula I wherein:

R¹=—O—C(═O)—CH═CH—R₅, R²=—CHR₄—CH₂—COON, R³=—(CH₂)₆—CH₃, R⁴=—(CH₂)₂—CH₃, and R⁵=—(CH₂)₆—CH₃;

R¹=α-L-rhamnopyranosyl substituted at the 2-position by —O—C(═O)—CH═CH—R⁵, R²=—CHR⁴—CH₂—COOCH₃, R³═(CH₂)₆—CH₃, R⁴=—(CH₂)₆—CH₃, and R⁵=—(CH₂)₆—CH₃;

R¹=—O—C(═O)—CH═CH—R₅, R²=—CHR₄—CH₂—COOCH₃, R³=—(CH₂)₆—CH₃, R⁴=—(CH₂)₂—CH₃, and R⁵=—(CH₂)₆—CH₃; and

R¹=α-L-rhamnopyranosyl substituted at the 2-position by —O—C(═O)—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.

In addition, the present invention relates to a method for treating a person or animal in need of a skin repair comprising the step of;

providing a composition comprising as an active ingredient a rhamnolipid of formula I,

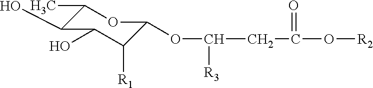

wherein R¹═H, unsubstituted α-L-rhamnopyranosyl, α.-L-rhamnopyranosyl substituted at the 2 position with a group of formula —O—C(═O)—CH═CH—R₅, or —O—C(═O)—CH═CH—R₅;
R²═H, lower alkyl, —CHR₄—CH₂—COOH or —CHR₄—CH₂—COOR₆;
R³=—(CH₂)ₓ—CH₃, wherein x=4-19;
R⁴=—(CH₂)_{y}—CH₃, wherein y=1-19;
R⁵=(CH₂)_{z}—CH₃, wherein z=1-12;
R⁶=lower alkyl;

applying the composition to the skin of the human or animal in an amount sufficient for the rhamnolipid to reach the bloodstream of the human or animal.

### BRIEF DESCRIPTION OF THE DRAWINGS

A more complete appreciation of the invention, and many of the attendant advantages thereof, will be readily obtained as the same becomes better understood by reference to the following detailed description when considered in connection with the accompanying drawings, wherein:

FIG. 1 illustrates a detail view of the different layers of the skin;

FIG. 2 shows a spectra showing the purity of the rhamnolipid according to the present invention;

FIG. 3 shows the results of the mass spectrograph of the rhamnolipid according to the present invention;

FIG. 4 shows the Fast atomic bombardment (FAB) mass spectra of the di-rhamnolipid according to the present invention;

FIG. 5a shows the bone narrow cells;

FIG. 5b shows the metaphases of the unthreaded bone narrow cells;

FIG. 5c shows the metaphases of the threaded bone narrow cells;

FIG. 6 shows the different stages of the treatment of dermatitis circumscripta unilocularis for an 80 year old female; and

FIG. 7 shows the development of the wound healing with 0.1% Di-rhamnolipid W3CTS eucerin ointment, day 0 (A), day 25 (B), and day 48 (C).

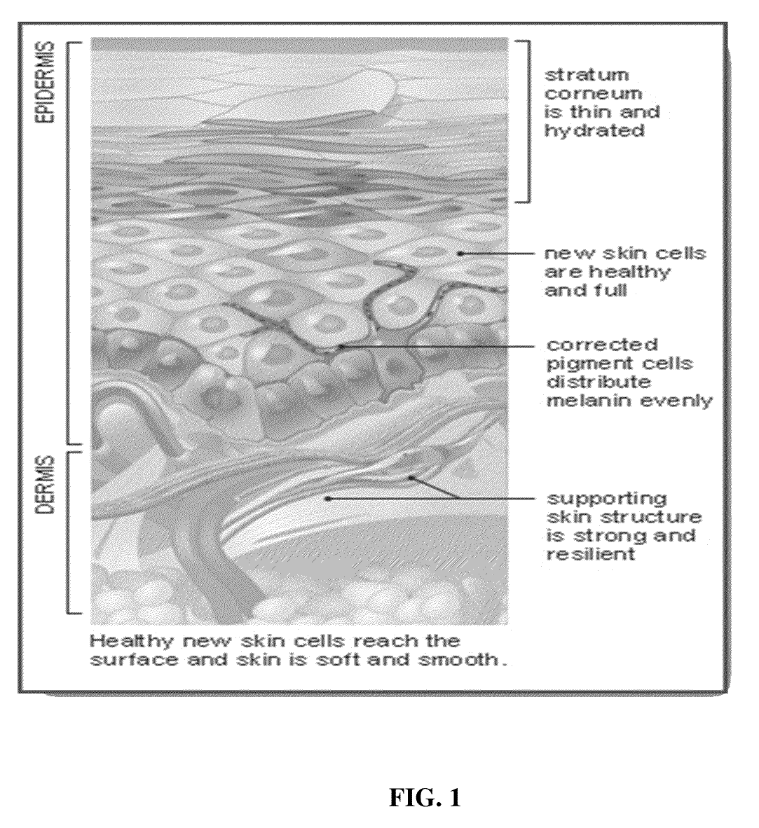

### DETAILED DESCRIPTION OF THE INVENTION

Recently, Applicant discovered that when the rhamnolipid according to the present invention is applied topically, the rhamnolipid is absorbed into the blood stream. In view of its small protean molecules, the rhamnolipid according to the present invention penetrates the epidermis of the skin, reaches the dermis, and enters into the blood stream, thereby delivering powerful repair properties through the entire body.

The enhanced blood stream increases the blood circulation in the micro-capillaries stimulating a faster cell turnover, further resulting in enhancement of the skin tissue. This enhanced blood stream also removes any irritation and inflammation from the immediate vicinity of an afflicted area.

The size of the small molecules of the rhamnolipid according to the present invention needs to be small enough to be metabolized in the bloodstream (i.e. absorbed by cells) and also sufficiently small enough so as not to cause blockage of the small capillaries in the circulatory system; thus, they must be less than about 1 micron.

The discovery that the rhamnolipids according to the present invention are capable of being distributed through the blood stream, opens the doors to a new technology that with a simple topical application of a composition having the rhamnolipid according to the present invention may treat at the same time different injuries, skin conditions, organ repairs, and/or wounds.

In addition, the present inventor discovered that because the rhamnolipid of the present invention attaches to the fibroblasts which produce collagen and make these cells proliferate at a more rapid rate than previously, which increases the structure and tensile strength of the skin from the deep layers. In the mature skin market, this discovery is a huge advance. It will smooth out and reduce the appearance of wrinkles within the deep layers of the skin. This is unprecedented in the skin care market, because the molecules of the formulations known in the art are too big to penetrate the epidermis and reach the dermis and the blood stream. Basically, the formulation known in the art only produces a film on the epidermis and cannot reach the deep layers of the skin.

This revolutionary discovery dramatically increases the production of collagen by as much as 30% with visible results seen in as little as one week. There is a significant reduction in the appearance of wrinkles, ruddiness and uneven skin texture, resulting in softer, smoother, younger looking and feeling skin in a relatively short time.

Furthermore, the present invention discovered that the rhamnolipids of the present invention are chemotactant, which means they attach to different molecules and are biodegradable in the body. They are effective at low concentration levels, are not known to cause toxicity to the human, and they are an effective sun protectant as well as increase the production of Vitamin D, which is a known antioxidant and anti-cancer vitamin. In addition, rhamnolipids have an exfoliating action on the skin which will remove the top dead layers of skin to reveal smoother, softer skin. It may be used in higher concentrations as a topical chemical peel.

In addition, the present inventor discovered that the rhamnolipid of the present invention also has an anti-bacterial, anti-inflammatory, and an antibiotic-like effect which is applicable in conditions such as acne, sunburn, and chemical or laser damaged skin. In a little higher concentration the rhamnolipids of the present invention are great wound healers, so in the Teenage/Acne skin care market, may be formulated for the client to see quantifiable results in faster healing of acne pustules as well as lessen the possibility of scarring.

Furthermore, the present inventor discovered that the rhamnolipid of the present invention possesses several properties which are beneficial for the treatment of mechanical skin wounds and burn wounds by accelerating the healing with less fibrosis and preventing bacterial inflammation and for normalizing some of the irradiated body dysfunction by its immunological specific activities.

The wound healing process involves a highly coordinated cascade of cellular responses encompassing the interaction of many cell types over long periods of time. The early phase of normal cutaneous wound healing is characterized by the influx of inflammatory cells from the circulation to the site of injury. Monocytes which become activated macrophages at the injured site, play multiple roles in wound healing, including release of proteases for wound debridement, phagocytosis of debris, and secretion of various cytokines and growth factors which, in turn, regulate the activity and interactions of other cell types involved in tissue repair.

Burn wounds, wounds from mechanical trauma and compressive injuries elicit different patterns of mortality from combined injuries. Previous research has shown that burning prior to, or concurrent with, radiation could reduce mortality and burning after radiation and could significantly increase mortality. The research design includes an animal model of full thickness skin burn wounds using Harlan Sprague-Dawley rats that were exposed 2 hours before burning to LD₅₀ or lower gamma radiation.

Based on in-vitro and in-vivo experimental results using the rhamnolipid of the present invention, it is possible to create a novel approach in the treatment of combined radiation injuries. Today, treatment of combined injuries is directed to separate targets in the body of combined radiation injured persons using different approaches for each organ.

A number of growth factors have not been effective in accelerating the healing of full-thickness burn wounds. For example, neither the platelet derived growth factor (PDGF) nor the epidermal growth factor (EGF) stimulated the closure. Wounds treated with EGF appeared to close rapidly (due to epithelialization), but reopened after the treatment was discontinued.

The present inventor conducted several studies to investigate the properties of the rhamnolipid of the present invention relating to the process of cutaneous wound healing after different doses of irradiation.

### Preliminary Data

### Production of Di-Rhamnolipid

Applicant has developed a method for production of Production of di-rhamnolipid by Ps. aeruginosa sp. After cultivation, the biomass was separated at Beckman Ultracentrifuge at 60,000 g, at room temperature. After removing biomass, the initial volume containing rhamnolipids was subjected to filtration through a 0.1 μm filter to remove the remaining bacteria and other debris. This step was followed by ultra filtration over 10⁶, and after that over 10⁵ molecular sieve exclusion Pellicon Cassettes (PTHK000C5) with Millipore continuous flow system (Millipore, Billerica, Mass., USA). Supernatant was collected and dissolved in the same amount of Millique filtered water and the whole procedure was repeated four times more. In this manner the color of fermentation broth was removed, leaving mostly discolored rhamnolipid pellets. After the final procedure, rhamnolipid pellets were collected and diluted in the distilled water to make up the initial volume. This solution contained dissolved rhamnolipids and was further processed using the adsorption chromatography system.

The initial low-pressure liquid chromatography was performed on adsorption resins Amberlite XAD-7 or XAD-8 (Rohm&Haas, Philadelphia, Pa., USA). The resins were prepared according to the standard procedure. The column 30×10 cm (Pharmacia) was filled by prepared XAD-7 or XAD-8 and equilibrated by 10 volumes of distilled water. The solution containing dissolved rhamnolipids was pumped through resin into the column. The flow rate was 4 column volumes per hour. The total capacity of the XAD-7 adsorption was for 30 liters of dissolved rhamnolipids. After the completion of adsorption on XAD-7 or XAD-8 resins, the column was washed with ten times higher volume of distilled water. The rhamnolipids were eluted with 50% EtOH with the same flow rate (4 column volumes per hour). The void volume was discharged and rhamnolipids were collected by fraction collector (Pharmacia) in half liter bottles. The first two bottles were containing mostly di-rhamnolipid (W3CTS) and the other bottles remaining contained rhamnolipids. All bottles were left overnight in the refrigerator at +4° C. Rhamnolipids were precipitated and supernatant was discharged. The precipitated rhamnolipid pellets were freeze-dried and used for further purification. Preparative and analytical HPLC was performed in reverse phase mode using the preparative and analytical waters system. The purity and mass spectrometry was determined.

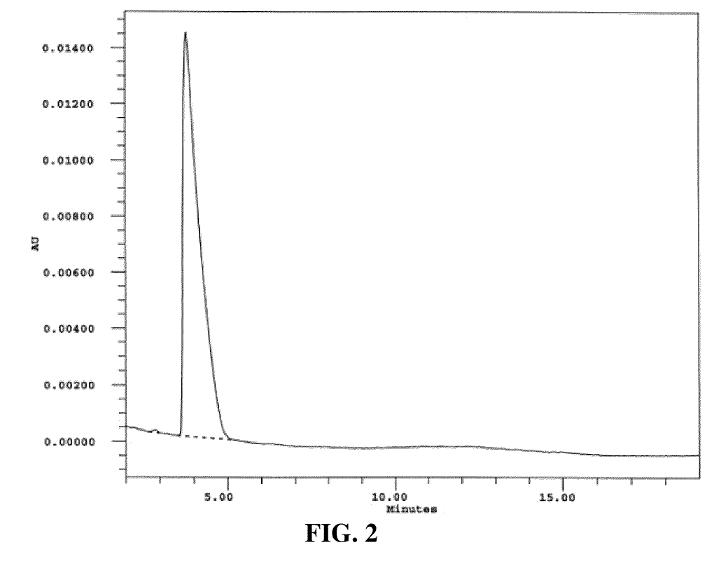

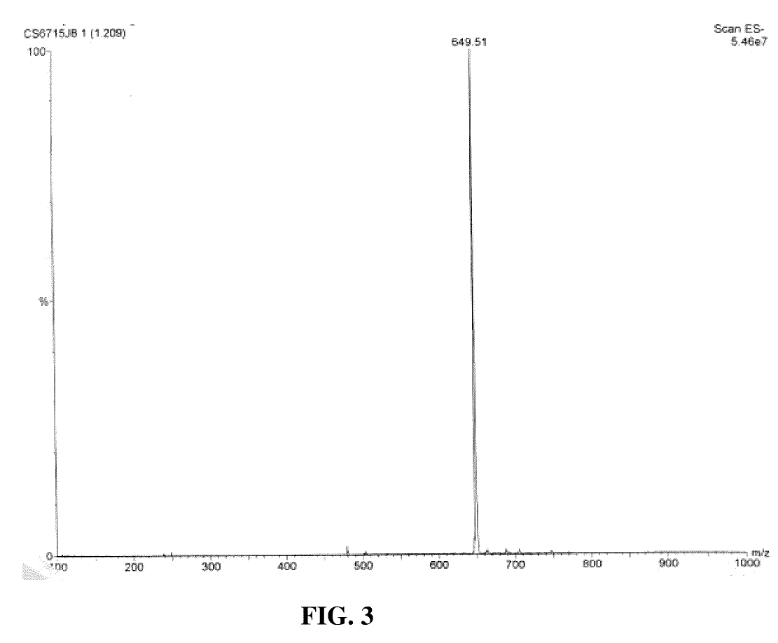

FIG. 2 shows the purity of the rhamnolipid according to the present invention. FIG. 3 shows the results of the mass spectrograph of the rhamnolipid according to the present invention.

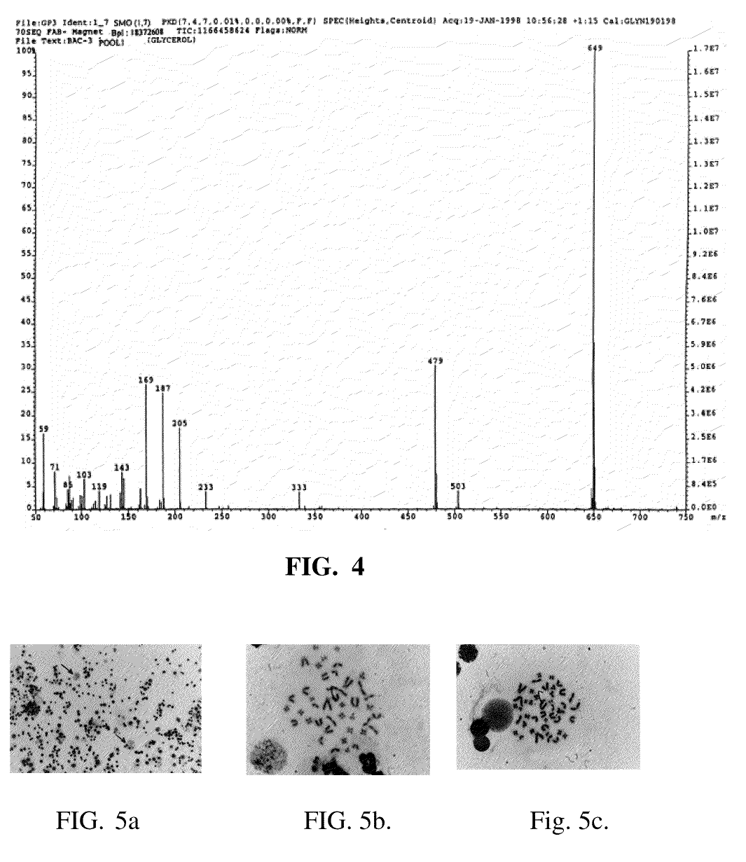

FIG. 4 shows the FAB spectrometry. Fast atomic bombardment (FAB) mass spectra of the di-rhamnolipid according to the present invention using Liquid Secondary Ion Mass Spectrometry/Collision Induced Dissociation (LSIMS/CID) on a VG70SEQ hybrid mass spectrometer (Micro mass, Manchester, UK) with EBQQ configuration equipped with cesium ion source.

The rhamnolipid according to the present invention may have one or more rhamnolipids of Formula I:

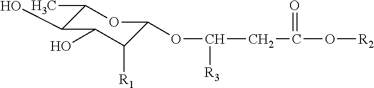

wherein R¹=H, unsubstituted α-L-rhamnopyranosyl, α.-L-rhamnopyranosyl substituted at the 2 position with a group of formula —O—C(═O)—CH═CH—R₅, or —O—C(═O)—CH═CH—R₅;
R²=H, lower alkyl, —CHR₄—CH₂—COOH or —CHR₄—CH₂—COOR₆;
R³=—(CH₂)ₓ—CH₃, wherein x=4-19;
R⁴=—(CH₂)_{y}—CH₃, wherein y=1-19;
R⁵=(CH₂)_{z}—CH₃, wherein z=1-12; and
R⁶=lower alkyl,

In one embodiment of the present invention, the rhamnolipid may be α-L-rhamnopyranosyl-(1,2)-α-L-rhamnopyranosyl)-3-hydroxydecanoyl-3-hydroxydecanoic acid having the following formula:

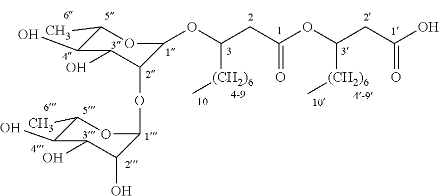

In another embodiment, in formula I R′ may be selected from:

R¹=—O—C(═O)—CH═CH—R₅, R²=—CHR₄—CH₂—COON, R³=—(CH₂)₆—CH₃, R⁴=—(CH₂)₂—CH₃, and R⁵=—(CH₂)₆—CH₃;

R¹=α-L-rhamnopyranosyl substituted at the 2-position by —O—C(═O)—CH═CH—R⁵, R²=—CHR⁴—CH₂—COOCH₃, R³═(CH₂)₆—CH₃, R⁴=—(CH₂)₆—CH₃, and R⁵═—(CH₂)₆—CH₃;

R¹=—O—C(═O)—CH═CH—R₅, R²=—CHR₄—CH₂—COOCH₃, R³=—(CH₂)₆—CH₃, R⁴=—(CH₂)₂—CH₃, and R⁵=—(CH₂)₆—CH₃; or

R¹=α-L-rhamnopyranosyl substituted at the 2-position by —O—C(═O)—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.

### In-Vitro Study with Di-Rhamnolipid

The rhamnolipid used for the study was α-L-rhamnopyranosyl-(1,2)-α-L-rhamnopyranosyl)-3-hydroxydecanoyl-3-hydroxydecanoic acid (hereafter called Di-rhamnolipid W3CTS) and has the formula

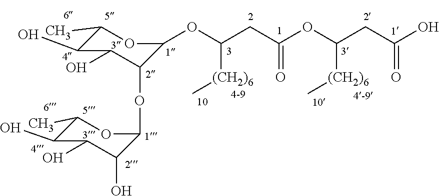

Di-rhamnolipid W3CTS has been isolated from Pseudomonas aeruginosa. Production of >98.5% pure Di-rhamnolipid W3CTS is a very expensive procedure; thus, the goal is to produce the clinically accepted purity of the rhamnolipid that has a long shelf life. The present inventor discovered that Di-rhamnolipid W3CTS kept under Nitrogen in bottles at room temperature did not change its structure during 10 years.

The present invention is not limited to the above indicated rhamnolipid and any rhamnolipid that meets the requirements of formula I is expected to produce the same results.

Colony forming efficiency of neonatal human keratinocytes treated with Di-rhamnolipid W3CTS.

At 100 μg/ml, 50 μg/ml, 10 μg/ml and 1 μg/ml concentrations, Di-rhamnolipid W3CTS stimulated the growth of keratinocyte colonies in GM medium with 10% fetal bovine serum. The stimulation was most significant at 100 μg/ml and 50 μg/ml the colony forming ability was increased by 34% at 50 μg/ml of Di-rhamnolipid W3CTS. The results are consistent with the effects of Di-rhamnolipid W3CTS on the neonatal human keratinocyte viability in GM with 10% fetal bovine serum.

### Neonatal Human Keratinocyte and Fibroblast Cell Cultures.

Di-rhamnolipid W3CTS at high concentration of 200 μg/ml and 500 μg/ml fully inhibited cell proliferation and viability, causing necrosis of neonatal human fibroblasts and keratinocytes.

On human neonatal keratinocytes grown without serum and at low calcium concentrations Di-rhamnolipid W3CTS fully inhibited proliferation and viability and caused necrosis at concentration as low as 100 μg/ml. On the contrary, Di-rhamnolipid W3CTS in the presence of serum and at high concentration of calcium increased the proliferation and the viability of keratinocytes and decreased the caspase activity. At the concentration of 50 μg/ml, Di-rhamnolipid W3CTS maximally stimulated the proliferation (34%) and the viability of keratinocytes (25%) as compared to control, and decreased the apoptosis (49%).

Di-rhamnolipid W3CTS in the presence of serum at the concentration 100 μg/ml maximally inhibited proliferation of human neonatal fibroblasts by 46% and viability by 28%. At the same concentration of 100 μg/ml W3CTS increased apoptosis by 72%.

Leukotriene C₄ Synthetase

Leukotriene C₄ (LTC₄) synthetase is involved in the formation of LTC₄ from LTA₄. Taken together with other assays for enzymes involved in the lipoxygenase pathway (e.g. 5-lipoxygenase, LTA₄ hydrolase), a locus of action can be established for agents which inhibit the formation of leukotrienes. Guinea pig lung LTC₄ synthetase was used. Test compound and/or vehicle was pre-incubated with 180 μg/ml enzyme in phosphate buffer pH 7.8 for 15 minutes at 37° C. The reaction is initiated by the addition of 2.5 μg/ml LTA₄ methyl ester for another 30 minute incubation period and terminated by further addition of ice-cold methanol. Determination of the amount of LTC₄ formed is read spectrophotometrically by enzyme immunoassay kit (EIA). W3CTS was screened at 1,000; 100; 10; and 1 μM/ml. Solvent was DMSO 0.5%. Sulfazine was used as a reference. Inhibition was 81% at the highest concentration of 1,000 μM/ml.

### Inhibition of Dopamine Receptors:

At the concentration of 100 μM Di-rhamnolipid W3CTS in 0.5% DMSO, Di-rhamnolipid W3CTS inhibited 100% dopamine D1 (human recombinant) binding of [³H] SCH23390. At the same concentration of 100 μM W3CTS in 0.5 DMSO measuring binding of [³H]Spiperone to human dopamine D_{2L} (human recombinant) was inhibited by 53%.

### Effects of Di-Rhamnolipid W3CTS on Mouse T-Cell and B-Cell Proliferation.

T-lymphocyte cells isolated from thymus of balb/c mice weighing 17±1 g were used. Test compound and/or vehicle was incubated with the cells (4×10⁶/ml) in the presence of 3 μg/ml concanavalin A (Con A) overnight. Thymidine incorporation was assessed by liquid scintillation counting. B-lymphocyte cells isolated from the spleen of balb/c mice weighing 17±1 g were used. Test compound and/or vehicle was incubated with the cells (1.5×10⁶/ml) in the presence of 10 μg/ml lipopolysaccharide (LPS) in AIM-V medium pH 7.4 at 37° C., during 24 hours. [³H]Thymidine (120 nM) was then added for an additional overnight incubation period. Thymidine incorporation was assessed by liquid scintillation counting. In both tests reference compound was cyclosporine A.W3CTS dissolved in 0.5% DMSO at the concentration of 10 μM did not have any effect on B-cell proliferation isolated from mouse spleen and inhibited proliferation of the same cells in the presence of LPS up to 54%. T-cells isolated from mouse thymus were inhibited at the concentration of 10 μM by 24% and in the presence of concanavalin T-cells proliferation was inhibited 43%.

In-Vivo Study with Di-Rhamnolipid W3CTS:

### Immunological Activities of Di-Rhamnolipid W3CTS

Immunostimulation (cellular): Oxazolone-induced delayed type hypersensitivity of female ICR mice was negative.

Immunosuppression (cellular): Oxazolone-induced delayed type hypersensitivity of female ICR mice was negative.

Immunostimulation (humoral) in BALB/c male mice and Immunosuppression (humoral)^{(2,7)} on ICR male mice were negative.

### Immunomodulation Activity.

Di-rhamnolipid W3CTS was administered i/p (100 mg/kg) to groups of 10 ICR derived male mice weighing 24±2 g at 24 hours before i/v challenge with a suspension of C. albicans (1×10⁷ cfu/mouse) sufficient to result in 90-100% mortality within 10 days in vehicle treated animals. Survival of 20 percent or more (≧20) of the animals is considered significant and indicates possible immunomodulation activity. Reference agent was lipopolysaccharide 0.3 mg/kg i/p. Test showed possible significant immunomodulation activity.

### Immunorestoration Activity.

Di-rhamnolipid W3CTS was administered i/p. (100 mg/kg) to groups of ICR derived male mice weighing 22±2 g on days 1, 3, and 5, with the immunosuppressant cyclophosphamide (30 mg/kg p/o.) administered on days 2, 4, and 6. One day after the last immunosuppressant dose, the mice were challenged with a suspension of C. Albicans sufficient to result in 90-100% mortality within 10 days in vehicle treated animals. Survival of 30 percent or more (≧30) of the animals was considered significant and indicated possible immunorestoration activity. Reference agent was lipopolysaccharide 0.3 mg/kg i/p. Test showed possible very significant immunorestoration activity.

Effects of Di-rhamnolipid W3CTS on TPA/pyr induced acute inflammation in the ears of Swiss-Webster female mice.

The aim of this study was two-fold. In Part A the potential of a single topical application of W3CTS to cause acute cutaneous irritation (up to 24 hours) was measured in a mouse ear assay system. No ear erythema was observed in the mice at any point up to 24 hours. Under these conditions, Di-rhamnolipid W3CTS did not cause acute cutaneous irritation, nor did the vehicle control. The aim of Part B was to determine the effects of a single topical administration of W3CTS on acute dermal inflammation induced by TPA/pyr, Part B was designed to differentiate the ability of Di-rhamnolipid W3CTS at concentration 1 mg/ml to prevent, inhibit or reverse induced ear inflammation. Overall, Di-rhamnolipid W3CTS at concentration (1 mg/ml, 1.0% w/v) was effective as a topical therapeutic anti-inflammatory agent in the TPA/pyr model of acute dermal ear inflammation in female Swiss-Webster mice over a time-course of 24 hours.

The time of maximal reduction of inflammation was dependent on the time of treatment, with respect to induction. The effect of Di-rhamnolipid W3CTS was moderate (up to 32% decrease in inflammation) but sustained. Di-rhamnolipid W3CTS was demonstrated to have a short duration as a protective agent when used as a pretreatment. Concurrent treatment effectively inhibited inflammation by about 32% at 6 hours. Post treatment reversed established inflammation by 32% at 11 hours. The results of Part B of this study suggest that topical Di-rhamnolipid W3CTS treatment can prevent induced swelling when given one hour prior to induced inflammation, inhibit inflammation when given concurrently and reverse inflammation when given 2 hours after inflammation has been induced. The effects were moderate in that swelling was not completely inhibited by W3CTS under any treatment conditions tested. It may be possible to increase the anti-inflammatory effect of Di-rhamnolipid W3CTS by use of a different dose formulation, or by repeated treatments.

Effects of Di-Rhamnolipid W3CTS on the Induction and Challenge Phases of DTH in Female Swiss-Webster Mice.

The aims of this study were first, to evaluate the in-vivo effects of Di-rhamnolipid W3CTS on aspects of a T-cell mediated immune response in mice and second, to determine if Di-rhamnolipid W3CTS was a sensitizing agent. This formed one of a series of studies to evaluate the toxicity and functionality of Di-rhamnolipid W3CTS.

The model used was induced delayed-type hypersensitivity (DTH) response in the mouse ear. In general, the DTH model involves administering a sensitizing agent mixed with an adjuvant, one or more occasions to prime the immune system. One to two weeks after the final induction and boost(s), animals are re-exposed to the sensitizing antigen, with a small volume injected intradermally in the ear, to elicit a secondary response (challenge). The ear swells during the secondary T-cell mediated response, and thus the change in ear thickness from prestimulation can be quantified. The present study had several sub-components testing different components of the immune response.

Parts A & B followed the standard procedure, in which the treatment test article, Di-rhamnolipid W3CTS, was administered topically concurrent to the challenge with sensitizing agent. Additionally, the effects of Di-rhamnolipid W3CTS on development of the primary immune response were also tested (Parts C&D). Therefore it was possible to obtain preliminary information about potential activity of Di-rhamnolipid W3CTS on more than one arm of the immune system. In parts B, C, and D, the protein ovalbumin, in CFA (Complete Freund's Adjuvant) or IFA (Incomplete Freund's Adjuvant) was used as the sensitizing agent as it is a standard with well-characterized, quantifiable activity in DTH. An additional compound, DNCB (1-chloro-2,4-di-nitro benzene) with gamma interferon (IFN-gamma), was also used (Group A) in order to maximize the opportunity to characterize the activity of Di-rhamnolipid W3CTS on T-cell mediated immunity. There have been proposals that DNCB and ovalbumin favor different TH arms of the T-cell response. To distinguish between an acute inflammatory response to W3CTS and DTH, the control condition was to administer saline at induction and boost, then to challenge with Di-rhamnolipid W3CTS.

In summary of Parts A-D, Di-rhamnolipid W3CTS did not show potential to enhance a primary immune response, when given at induction (Part D). On the other hand (Part C), when Di-rhamnolipid W3CTS was concurrently administered at the time of induction of a moderate immune response (ovalbumin/IFA), it was found to inhibit the subsequent DTH response. In Part B, which tested the effect of Di-rhamnolipid W3CTS on the secondary immune response generated by ovalbumin/CFA, W3CTS appeared to cause a sustained or increased DTH response, while a standard corticosteroid immunosuppressive agent gave the expected inhibition of DTH. Finally in Part A, which had a similar objective to Part B, but used a different induction agent, no DTH was elicited, and hence it did not allow Di-rhamnolipid W3CTS to be evaluated. Therefore it appears a difference in the effects of Di-rhamnolipid W3CTS on the primary and secondary immune response in mice ear DTH. As these processes have different mechanisms of action, there may be a fundamentally different action of Di-rhamnolipid W3CTS in modulating these different parts of the T-cell immune response.

### Effects of Di-Rhamnolipid W3CTS on DTH in Nude Guinea Pigs.

In-vitro studies showed that Di-rhamnolipid W3CTS is likely to work through local immune suppression, the classical hapten induced delayed type hypersensitivity model of contact dermatitis is an appropriate model. The nude guinea pig is used as a test animal because of different animal skins; nude guinea pig skin is claimed to be the one closest resembling human skin.

In DNCB sensitized guinea pigs delayed type hypersensitivity reactions were induced with patches soaked with 0.25% (w/v) DNCB in ethanol/propylene glycol kept on the animals back for 24 hours. The test sites were treated twice daily during 7 days with: Dovonex ointment (steroid), 1% Di-rhamnolipid W3CTS in Vaseline, vehicle and untreated area. By day 7^{th} the Dovonex ointment and the Di-rhamnolipid W3CTS test areas were healed with only a slight erythema present at place treated with Dovonex, while untreated and the vehicle treated areas still had a substantial erythema and indurations. Dovonex as well as vehicle induced inflammation in the surrounding skin. Dovonex has been improved by time. The model was not optimized completely yet, but preliminary data from guinea pigs showed a very strong anti-inflammatory effect of Di-rhamnolipid W3CTS in this model.

Maximum tolerated doses of di-rhamnolipid W3CTS were administered intravenously in female Swiss-Webster mice.

Female Swiss-Webster mice obtained from the Houston, Tex. site Harlan Sprague Dawley, Inc were used in the study. All mice were divided into 6 groups. The vehicle and Di-rhamnolipid W3CTS were administered as an intravenous bolus, via tail vein, beginning with lowest dose level (75 mg/kg). Surviving animals were observed for clinical signs of toxicity and body weights were monitored periodically. All mice in the 0, 75, 90, 105, and 120 mg/kg groups survived dosing and the 14 day follow-up period. Except for the tail injection site lesions on some animals in the highest dose groups, no gross abnormalities were seen in any animal necropsy. In this study, the maximum tolerated dose of di-rhamnolipid W3CTS in female Swiss-Webster mice was 120 mg/kg. Three mice (LD₅₀) of the next higher dose tested (135 /mg/kg) died immediately upon administration. Surviving animals tolerated Di-rhamnolipid W3CTS well, with the exception of the purple discoloration of the tails related to the site injection site. This condition resolved in all mice in the lower dose groups 75, 90 & 105 mg/kg dose groups, however scabbing and necrosis of tails was found in higher dose groups (120 mg/kg).

Blood findings in female and male mice treated i/v with one single dose of 75 mg/kg of Di-rhamnolipid W3CTS

Fourth day after application neutrophils were increased in blood of treated animals by 51.77% and after 29 days were still increased by 43.61%. At the same time, mice lymphocytes were decreased by 24.15%. Twenty nine days after injection neutrophils were still increased by 43.61% and lymphocytes decreased by 24.60%. Monocytes were increased in treated mice up to 100% after 4^{th} day and 29^{th}

Multi-dose toxicity study of Di-rhamnolipid W3CTS administered subcutaneously in female Swiss-Webster mice for 7 days recovery period. The effects of 7 daily subcutaneous injections of the 99.3% pure Di-rhamnolipid W3CTS, followed by 7-day recovery period, were determined in female Swiss-Webster mice. Five groups (3 mice per group) were administered W3CTS doses at 0.012; 1, 2; 60 and 120 mg/kg/day and vehicle. All animals were observed and weighed throughout the study for clinical signs of toxicity including skin injection sites. On study Day 13 (seven days after last injection, 6 days for group 5) all animals were euthanized. The thymus, liver, kidneys, spleen, heart and lungs were weighed and preserved.

No mortality occurred at any dose level. There were no significant differences in the body weights of any of the groups. Abnormal clinical observations at some animals at groups (60 and 120 mg/kg) were limited to skin lesions at the injection sites. All skin lesions were healing at termination.

At necropsy, two animals from group 120 mg/kg had moderate enlargement of the spleen and liver and adhesions between liver, spleen, kidneys and peritoneum were seen also in one animal (See Table 1).

TABLE 1

| | | DOSE | | | |
|---|---|---|---|---|---|
| GROUP | TEST | LEVEL | NO/ | | |
| NO. | ARTICLE | (mg/kg) | GROUP | MORTAL. | SIGNIFICANT FINDINGS |
| 1 | Vehicle | 0 | 3 | 0/3 | None |
| | DPBS | | | | |
| 2 | W3CTS | 0.012 | 3 | 0/3 | None |
| 3 | W3CTS | 1.20 | 3 | 0/3 | None |
| 5 | W3CTS | 60 | 3 | 0/3 | Injection site: skin lesions started on day |
| | | | | | 5. 1/3 mice with 1/7 injection site, 2/3 |
| | | | | | mice with 2/7 injection site on termination |
| | | | | | day |
| 4 | W3CTS | 120 | 3 | 0/3 | Injection site: skin lesions (3/3 mice) |
| | | | | | started on Day 1, affecting 6/7 injection |
| | | | | | sites, were still apparent but healing on |
| | | | | | termination day. |
| | | | | | Necroscopy findings: moderate |
| | | | | | enlargement of spleen and liver 2/3 mice; |
| | | | | | adhesions between the liver and the |
| | | | | | spleen, kidneys and peritoneum (1/3 |
| | | | | | mice). |
| | | | | | Organ Weights: liver and spleen weights |
| | | | | | slightly higher than all other groups. |
| | | | | | Thymus slightly lower in weight than all |
| | | | | | other groups (not statistically significant) |
| | | | | | Histopathology: thymic involution in 1 |
| | | | | | mouse, extramedullary hematopoiesis in 2 |
| | | | | | mice. Skin lesions showed chronic |
| | | | | | inflammation, progressing ulceration, |
| | | | | | occasional acanthosis, hyperkeratinosis & |
| | | | | | loss of adnexal structures. Appeared to |
| | | | | | be healing, possibly normally with more |
| | | | | | time. |

Histopathology of spleen, thymus, and liver and selected skin site lesions was performed on tissues from high dose group animals and one control. Abnormal findings of thymic involution, which correlated with slightly lower organ weight, were noted in one mouse and extramedullary hematopoiesis was noted in two mice. Histopathologic features of skin lesions included evidence of chronic active inflammation, co-existing with progressing ulceration and/or healing.

The effects of Di-rhamnolipid W3CTS on full-thickness burn wounds in normal Sprague-Dawley rats.

Di-rhamnolipid W3CTS was prepared in a eucerine ointment applied topically on full-thickness burn wounds in normal Sprague-Dawley rats covering 5% of the whole skin area. The rate of wound closure was measured over the period of 45 days. The collagen content was evaluated microscopically, by performing densitometric analysis on Verhoeff's stained histopathological slides of wound biopsies taken at the end of 45th day of di-rhamnolipid treatment. The treatment of full thickness-burn wounds covering 5% of the whole body skin area with topical applications (2 per day) of 0.1% di-rhamnolipid in eucerine ointment accelerated the closure of wounds on day 21 of the treatment by 32% compared to the control (p<0.05). On day 35, the wounds closed in all animals treated with 0.1% di-rhamnolipid ointment while some rats in the control group had open wounds on days 35 and even 45. Histological comparisons have shown that the same di-rhamnolipid W3CTS concentration (0, 1% in eucerine) significantly decreased the collagen content in burn wounds (47.5%, p<0.05) as compared to the vehicle treated (control) wounds. The results of this study raise the possibility of potential efficacy of di-rhamnolipid in accelerating normal wound healing and perhaps in overcoming defects associated with healing failure in chronic wounds.

Test on Mutagenesis In-Vivo:

Five days old Sprague Dawley animals, 200-250 g in mass, were injected with 31.5 mg of testing preparations per one kilogram of body weight, while the control group received only physiological solution of the same volume as the tested group.

The results of analysis on the chromosomal structural aberrations in the rat bone marrow cells show that the concentration of the BAC substance of 31.5 μg per gram of the animal body weight did not cause damage. In the control and treated samples there were some individual breaks of the chromosomes and chromatides accompanied with acentric fragments in only 1% of the analyzed cells. Such results could be accepted as a technical error and not as an interaction of the BAC substance with the bone marrow DNA.

FIG. 5a shows the bone narrow cells. FIG. 5b shows the metaphases of the unthreaded bone narrow cells. FIG. 5c shows the metaphases of the threaded bone narrow cells.

### Physician Sponsored Treatment of Psoriatic Patients

Pure 98.7% Di-rhamnolipid W3CTS was tested against A431 cells in keratinocyte growth medium with and without the presence of serum. Di-rhamnolipid W3CTS showed different effects that were later confirmed on human neonatal keratinocytes culture. When Ames test and micronucleus test with Di-rhamnolipid W3CTS in-vivo were shown to be negative, the patients were treated with Di-rhamnolipid W3CTS in 1.0% eucerin ointment.

Twelve psoriatic hospital bed patients, and one patient with seborrheic dermatitis and psoriasis, one patient with lichen planus and one with dermatitis cicumscripta unilocullaris as an out clinic patient were recruited as study volunteers. The results and follow up of these twelve psoriatic patients were presented on Table 2.

TABLE 2

| | Patient | Diagnosis | | Psoriasis Area Severity Index | | | | | |
|---|---|---|---|---|---|---|---|---|---|
| | (sex, | (affliction | Treating | Before Treatment | | After Treatment | | Extended Evaluation | |
| | age) | & duration) | days | W3CTS | Ster. | W3CTS | Ster. | W3CTS | Ster. |
| 1 | M 31 y | PVP 10 y | 21 d | 8.1 | 5.4 | 0.6 | 3.0 | 3 m relapse | 3 m relapse |
| 2 | M 57 y | PVP 18 m | 21 d | 8.4 | 8.4 | 1.6 | 6.0 | 3 m = 0.1 | 3 m relapse |
| 3 | M 35 y | PVP 2.5 y | 21 d | 9.6 | 10.8 | 0.2 | 8.4 | N/a | N/a |
| 4 | M 41 y | PVP 19 y | 21 d | 8.1 | 8.1 | 0.9 | 7.2 | 3 m = 0..2 | 3 m relapse |
| 5 | M 30 y | PVP 7 y | 21 d | 9.6 | 13.2 | 0.2 | 10.8 | 3 m = 0 | 3 m relapse |
| 6 | M 49 y | PVP 10 y | 15 d | 8.1 | 8.1 | 0.3 | 0.3 | N/a | N/a |
| 7 | M 47 y | PVP 23 y | 40 d | 10.8 | 6.3 | 0.4 | 2.4 | 1 y = 0 | 1 y relapse |
| 8 | M 47 y | PVP 5 y | 21 d | 10.8 | 9.6 | 0.2 | 3.6 | N/a | N/a |
| 9 | F 44 y | PVP 23 y | 36 d | 10.8 | 6.0 | 0.2 | 0.4 | 1 y = 0 | 1 y relapse |
| 10 | F 68 y | PVP 7 y | 40 d | 10.8 | 5.4 | 6 | 3.0 | 1 y = 0 | 1 y relapse |
| 11 | M 46 y | PVP 21 y | 21 d | 14.4 | 10.8 | 0.6 | 3.6 | 3 m = 0 | 3 m relapse |
| 12 | F 30 y | PVP 7 y | 21 d | 5.4 | 5.4 | 0.2 | 0.2 | 1 y = 0 | 1 y relapse |

Abbreviations: PASI=Psoriasis Area Severity Index (Erythema, Infiltration and Scaling); PVP=Psoriasis Vulgaris in Placibus; d=days; m=months; y=years; ster=steroids (treated)

Some of the patients were free of psoriatic lesions up to seven years. Patients with seborhheic dermatitis, lichen planus and dermatitis unilocularis circumscripta eczematoides were cured.

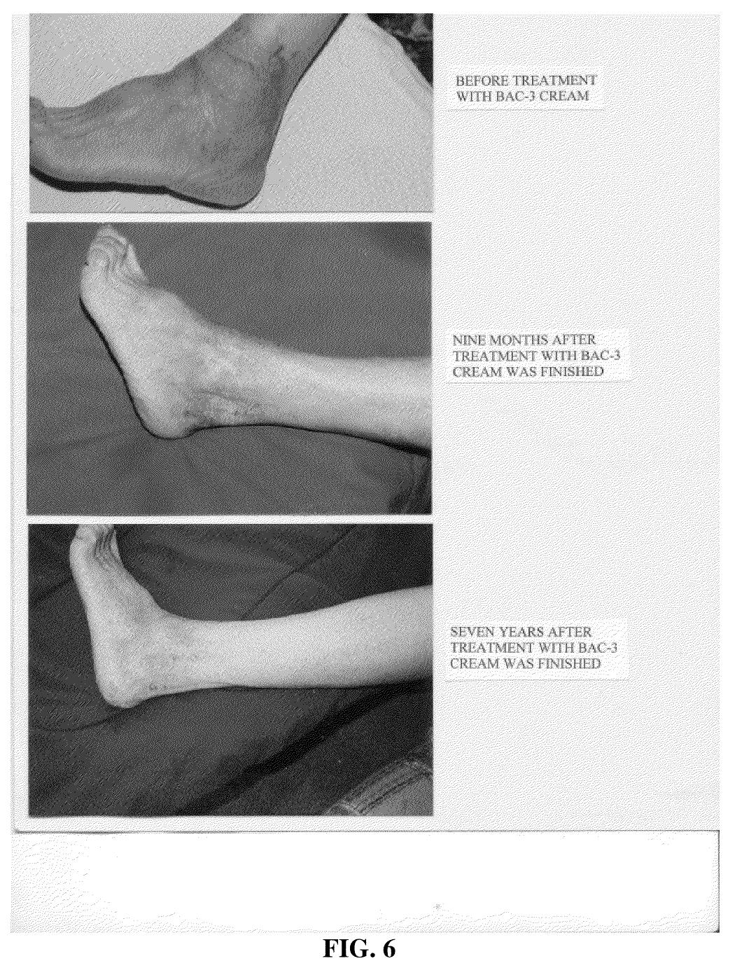

FIG. 6 shows the different stages of the treatment of dermatitis circumscripta unilocularis for an 80 year old female. This condition, confirmed with clinical appearance and histopahological findings, was treated with different standard approaches, (mostly) steroids, but condition worsened with tendency to spread further to the large wound covered by thick crust. Following the treatment with 1% Di-rhamnolipid W3CTS, eucerin ointment was introduced with overnight inclusions during four weeks. The wound started to heal and the whole process is presented in pictures taken on the 1^{st} day of the treatment with 1% Di-rhamnolipid W3CTS in eucerin ointment and following up to the next seven years. After one year the skin over the wound was looking normal. Patient died by natural course 15 years after treatment had been finished, at age 93.

Treatment of Chronic Decubitus Ulcer with Di-Rhamnolipid W3CTS

This case demonstrates that the topical Di-rhamnolipid W3CTS can have a profound beneficial effect on the healing of chronic decubitus ulcer in humans. The mechanism of action of the Di-rhamnolipid W3CTS lies in the demonstrated biological effects of Di-rhamnolipid W3CTS in facilitating wound re-epithelization and remodeling through immune system interaction.

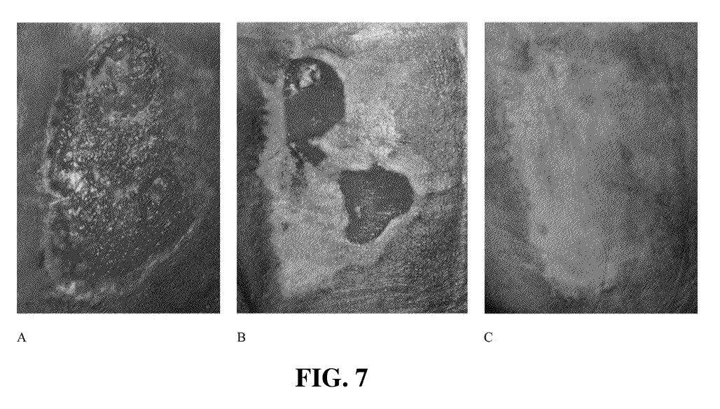

FIG. 7 shows the devolopment of the wound healing with 0.1% Di-rhamnolipid W3CTS eucerin ointment, day 0 (A), day 25 (B), and day 48 (C).

The mechanism of action of Di-rhamnolipid W3CTS is important for the process of wound healing: stimulation of bone marrow to produce significantly more neutrophils and monocytes and inhibit the production of lymphocytes and chemoattractant activity of Di-rhamnolipid W3CTS for neutrophils at the place of application is a property of the dirhamnolipid that is particularly important at the inflammatory stage of wound healing. During this phase, fast recruitment of polymorphonuclear leukocytes is necessary for removal of bacteria, foreign particles, and de-briding tissue and for the release of cytokines and growth factors necessary for directing fibroblasts, keratinocytes, and endothelial cells to repair the damaged blood vessels. The detergent activity of Di-rhamnolipid W3CTS is useful since it could act to dissolve a biofilm created by colonized bacteria in wounds, providing access to neutrophils and macrophages, which de-bride the wound. Di-rhamnolipid W3CTS stimulation of the proliferation of keratinocytes further assists the wound reepithelialization, consequently producing more growth factors, chemoattractants, and proteases available to dissolve the rest of the nonviable tissue. Lastly, Di-rhamnolipid W3CTS diminution of fibrosis is a property of dirhamnolipid that might justify its potential application in the treatment of not only chronic wounds but also other conditions associated with increased fibroblast proliferation (scarring).

Those of skill in the art will recognize that other additives may also be added to the Rhamnolipid formulations to increase the effectiveness of the overall product, or process.

What is claimed is:

1. A composition for treating a patient comprising as active ingredient a rhamnolipid of formula I, 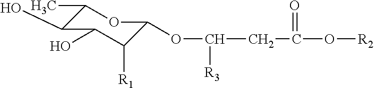
wherein R¹═H, unsubstituted α-L-rhamnopyranosyl, α.-L-rhamnopyranosyl substituted at the 2 position with a group of formula —O—C(═O)—CH═CH—R₅, or —O—C(═O)—CH═CH—R₅;
R²═H, lower alkyl, —CHR₄—CH₂—COOH or —CHR₄—CH₂—COOR₆;
R³=—(CH₂)ₓ—CH₃, wherein x=4-19;
R⁴=—(CH₂)_{y}—CH₃, wherein y=1-19;
R⁵═(CH₂)_{z}—CH₃, wherein z=1-12;
R⁶=lower alkyl;
wherein the composition is a topical composition that is absorbed by the skin of a human or animal, absorbed by the blood stream, and distributed through the human or animal body by the blood stream.

2. The composition according to claim 1, wherein rhamnolipid of formula I has a concentration of between 1-100 μg/ml.

3. The composition according to claim 1, wherein rhamnolipid of formula I has molecules having a size of about 1 micron.

4. The composition according to claim 1, wherein the rhamnolipid of Formula 1 is α-L-rhamnopyranosyl-(1,2)-α-L-rhamnopyranosyl)-3-hydroxydecanoyl-3-hydroxydecanoic acid having the following formula:

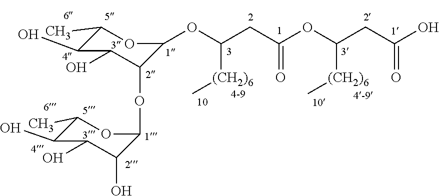

5. The composition according to claim 1, wherein the one or more rhamnolipids of Formula I are selected from the group consisting of compounds of Formula I wherein:
R¹=—O—C(═O)—CH═CH—R₅, R²=—CHR₄—CH₂—COON, R³=—(CH₂)₆—CH₃, R⁴=—(CH₂)₂—CH₃, and R⁵=—(CH₂)₆—CH₃;
R¹=α-L-rhamnopyranosyl substituted at the 2-position by —O—C(═O)—CH═CH—R⁵, R²=—CHR⁴—CH₂—COOCH₃, R³═(CH₂)₆—CH₃, R⁴=—(CH₂)₆—CH₃, and R⁵=—(CH₂)₆—CH₃;
R¹=—O—C(═O)—CH═CH—R₅, R²=—CHR₄—CH₂—COOCH₃, R³=—(CH₂)₆—CH₃, R⁴=—(CH₂)₂—CH₃, and R⁵=—(CH₂)₆—CH₃; and
R¹=α-L-rhamnopyranosyl substituted at the 2-position by —O—C(═O)—CH═CH—R⁵, R²=—CHR⁴—CH₂—COOCH₃, R³=—(CH₂)₆—CH₃, R⁴=—(CH₂)₆—CH₃, and R⁵=—(CH₂)₆—CH₃.

6. The composition according to claim 1, wherein the composition is an anti-inflammatory composition and wherein the rhamnolipid of formula I reduces or inhibits the inflammation.

7. The composition according to claim 1, wherein the composition is a wound healing composition and wherein wound epithelization and remodeling is through the absorption of the composition by the blood stream.

8. The composition according to claim 1, wherein the rhamnolipid of formula I attaches to fibroblasts increasing the production of collagen.

9. A method for treating a person or animal in need of a skin repair, the method comprising the step of;
a) providing a composition comprising as an active ingredient a rhamnolipid of formula I, 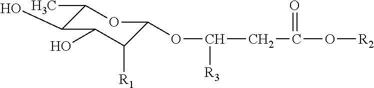
wherein R¹═H, unsubstituted α-L-rhamnopyranosyl, α.-L-rhamnopyranosyl substituted at the 2 position with a group of formula —O—C(═O)—CH═CH—R₅, or —O—C(═O)—CH═CH—R₅;
R²═H, lower alkyl, —CHR₄—CH₂—COOH or —CHR₄—CH₂—COOR₆;
R³=—(CH₂)ₓ—CH₃, wherein x=4-19;
R⁴=—(CH₂)_{y}—CH₃, wherein y=1-19;
R⁵═(CH₂)_{z}—CH₃, wherein z=1-12;
R⁶=lower alkyl;
b) applying the composition to the skin of the human or animal in an amount sufficient for the rhamnolipid to reach the bloodstream of the human or animal.
